(19) United States
(12) Patent Application Publication
(10) Pub. No.: US 2024/0173488 A1
(43) Pub. Date: May 30, 2024

(54) VIBRATING ANESTHESIA DEVICE

(71) Applicant: SUPERIOR DERMATOLOGY SOLUTIONS LLC, Conroe, TX (US)

(72) Inventor: Rod Mahmoudizad, Conroe, TX (US)

(21) Appl. No.: 18/525,163

(22) Filed: Nov. 30, 2023

Related U.S. Application Data
(60) Provisional application No. 63/385,616, filed on Nov. 30, 2022.
(60) Provisional application No. 63/387,317, filed on Dec. 14, 2022.

(51) Int. Cl. A61M 5/42

(52) U.S. Cl. CPC A61M 5/422; A61M 2210/083

(57) ABSTRACT

A vibrating anesthesia device with finger cuffs, a pressure switch, and a control unit. The finger cuffs are configured to fit onto the fingers of the user and each may have a vibrator motor positioned adjacent to the fingertip when the finger cuff is worn. The pressure switch is positioned within one of the finger cuffs and is configured to sense a force exerted on the pressure switch. The control unit is configured to attach to the wrist of the user and is electrically coupled to the pressure switch and to each of the vibrator motors. The control unit provides power to the pressure switch, monitors the force exerted on the pressure switch, and turns on each of the vibrator motors when the force exerted on the pressure switch exceeds a threshold value. When turned on, the vibrator motors vibrate and can provide a local anesthetic effect to a patient.

### RELATED APPLICATIONS

This application claims the benefit of the filing date of U.S. Provisional Patent Application No. 63/385,616, titled “Vibrating Anesthesia Device,” to Dr. Rod Mahmoudizad, filed Nov. 30, 2022, as well as U.S. Provisional Patent Application No. 63/387,317, titled “Vibrating Anesthesia Device,” to Dr. Rod Mahmoudizad, filed Dec. 14, 2022, the entirety of the disclosures of which are hereby incorporated by this reference.

### TECHNICAL FIELD

This document relates to a vibrating anesthesia device for reducing pain at a medical procedure site during a medical procedure, such as when inserting a needle, drawing blood, or during other medical procedures.

### BACKGROUND

Standard methods for patient care during minor medical procedures that do not require anesthesia, such as during needle insertion, include holding a patient's hand or distracting the patient. Such methods fail to decrease pain and may lead to decreased patient satisfaction with the patient believing that the medical professional is uncaring. While some devices exist that implement vibrations to reduce pain during medical procedures, these are inconvenient for medical professionals and are therefore not typically implemented.

### SUMMARY

Aspects of this document relate to a vibrating anesthesia device, comprising at least two flexible finger cuffs each configured to fit onto a finger of a user, each of the at least two finger cuffs having a vibrator motor configured to be positioned adjacent a fingertip of the user when the finger cuff is positioned on the finger of the user, wherein a first finger cuff of the at least two finger cuffs is sized and shaped to fit on an index finger of the user and a second finger cuff of the at least two finger cuffs is sized and shaped to fit on a thumb of the user, a pressure switch positioned within the first finger cuff, wherein the pressure switch is configured to sense a force exerted on the pressure switch, and a control unit configured to attach to a wrist of the user, the control unit having a power source, a controller, and an interface configured to allow the user to control the vibrating anesthesia device, wherein the control unit is electrically coupled to the pressure switch and to each of the vibrator motors and wherein the control unit is configured to provide power to the pressure switch, monitor the force exerted on the pressure switch, and turn on each of the vibrator motors of the at least two finger cuffs when the force exerted on the pressure switch exceeds a threshold value, wherein, when turned on, the vibrator motors are configured to vibrate and provide a local anesthetic effect to a patient.

Particular embodiments may comprise one or more of the following features. The vibrating anesthesia device may further comprise a glove configured to fit over a hand of the user and create a barrier between the vibrating anesthesia device and the patient, wherein an index finger of the glove is sized to fit the first finger cuff and the index finger of the user, and a thumb of the glove is sized to fit the second finger cuff and the thumb of the user. The control unit may be configured to turn on each of the vibrator motors only after the force exerted on the pressure switch exceeds the threshold value for at least two seconds. The vibrator motors may be configured to vibrate at a frequency between 100 Hz and 200 Hz. The threshold value may be at least 3.5 Newtons.

Aspects of this document relate to a vibrating anesthesia device, comprising a first finger cuff configured to fit onto a finger of a user, the first finger cuff having a vibrator motor configured to be positioned adjacent a fingertip of the user when the finger cuff is positioned on the finger of the user, a pressure switch positioned within the first finger cuff, wherein the pressure switch is configured to sense a force exerted on the pressure switch, and a control unit configured to attach to a wrist of the user, wherein the control unit is electrically coupled to the pressure switch and to the vibrator motor and wherein the control unit is configured to provide power to the pressure switch, monitor the force exerted on the pressure switch, and turn on the vibrator motor when the force exerted on the pressure switch exceeds a threshold value, wherein, when turned on, the vibrator motor is configured to vibrate and provide a local anesthetic effect to a patient.

Particular embodiments may comprise one or more of the following features. The vibrating anesthesia device may further comprise a second finger cuff configured to fit onto a second finger of the user, the second finger cuff having a vibrator motor, wherein the control unit is configured to turn on the vibrator motor of the second finger cuff with the vibrator motor of the first finger cuff. The first finger cuff may be sized and shaped to fit on an index finger of the user and the second finger cuff may be sized and shaped to fit on a thumb of the user. The vibrating anesthesia device may further comprise a glove configured to fit over a hand of the user and create a barrier between the vibrating anesthesia device and the patient, wherein an index finger of the glove is sized to fit the first finger cuff and the index finger of the user, and a thumb of the glove is sized to fit the second finger cuff and the thumb of the user. The control unit may have a power source, a controller, and an interface configured to allow the user to control the vibrating anesthesia device. The control unit may be configured to turn on the vibrator motor only after the force exerted on the pressure switch exceeds the threshold value for at least two seconds. The vibrator motor may be configured to vibrate at a frequency between 100 Hz and 200 Hz. The threshold value may be at least 3.5 Newtons.

Aspects of this document relate to a vibrating anesthesia device, comprising one or more finger cuffs each configured to fit onto a finger of a user, at least one of the one or more finger cuffs having a vibrator motor configured to be positioned adjacent a fingertip of the user when the finger cuff is positioned on the finger of the user, a pressure switch positioned within one of the one or more finger cuffs, wherein the pressure switch is configured to sense a force exerted on the pressure switch, and a control unit electrically coupled to the pressure switch and to each of the vibrator motors, wherein the control unit is configured to provide power to the pressure switch, monitor the force exerted on the pressure switch, and turn on each of the vibrator motors of the one or more finger cuffs when the force exerted on the pressure switch exceeds a threshold value, wherein, when turned on, the vibrator motors are configured to vibrate and provide a local anesthetic effect to a patient.

Particular embodiments may comprise one or more of the following features. The one or more finger cuffs may comprise a first finger cuff configured to fit onto a first finger of the user and a second finger cuff configured to fit onto a second finger of the user, wherein each of the first finger cuff and the second finger cuff has a vibrator motor configured to turn on when the force exerted on the pressure switch exceeds the threshold value. The first finger cuff may be sized and shaped to fit on an index finger of the user and the second finger cuff may be sized and shaped to fit on a thumb of the user. The vibrating anesthesia device may further comprise a glove configured to fit over a hand of the user and create a barrier between the vibrating anesthesia device and the patient, wherein an index finger of the glove is sized to fit the first finger cuff and the index finger of the user, and a thumb of the glove is sized to fit the second finger cuff and the thumb of the user. The control unit may have a power source, a controller, and an interface configured to allow the user to control the vibrating anesthesia device. The control unit may be configured to turn on the vibrator motor only after the force exerted on the pressure switch exceeds the threshold value for at least two seconds. The vibrator motor may be configured to vibrate at a frequency between 100 Hz and 200 Hz. The threshold value is at least 3.5 Newtons.

Aspects of this document relate to a method of reducing pain during a medical procedure, comprising selecting a medical procedure site on a patient, applying a force to the patient adjacent to the medical procedure site, applying a vibration adjacent to the medical procedure site in response to the force rising above a threshold value, and performing the medical procedure at the medical procedure site.

Particular embodiments may comprise one or more of the following features. The method may further comprise maintaining the vibration adjacent to the medical procedure site for at least 15 seconds. The method may further comprise measuring a length of time that the force remains above the threshold value and only applying the vibration adjacent to the medical procedure site after the force remains above the threshold value for at least two seconds. Applying the vibration adjacent to the medical procedure site may comprise applying the vibration at two points adjacent to the medical procedure site. The method may further comprise applying the vibration at a first point of the two points in a first direction and applying the vibration at a second point of the two points in a second direction different from the first direction. The medical procedure site may be positioned between the two points. The method may further comprise stopping the vibration when the force lowers past the threshold value. The vibration may have a frequency between 100 Hz and 200 Hz. The threshold value may be at least 3.5 Newtons.

Aspects of this document relate to method of using a vibrating anesthesia device, comprising inserting a first finger of a user into a first finger cuff having a first motor, inserting a second finger of the user into a second finger cuff having a second motor, contacting a patient adjacent to an medical procedure site on the patient with the first finger cuff and with the second finger cuff, turning on the first motor and the second motor, vibrating the medical procedure site with the first motor and with the second motor, performing the medical procedure at the medical procedure site, and turning off the first motor and the second motor.

Particular embodiments may comprise one or more of the following features. The method may further comprise attaching a control unit to a wrist of the user. Vibrating the medical procedure site may comprise vibrating the medical procedure site for at least 15 seconds. Vibrating the medical procedure site may comprise vibrating the medical procedure site at a frequency between 100 Hz and 200 Hz. The first finger may be an index finger. The second finger may be a thumb. Turning on the first motor and the second motor may be in response to applying a force above a threshold value to the patient adjacent to the medical procedure site. The threshold value may be at least 3.5 Newtons. Contacting the patient adjacent to the medical procedure site may comprise positioning the medical procedure site between the first finger cuff and the second finger cuff. The method may further comprise inserting a hand of the user into a glove.

Aspects of this document relate to method of reducing pain during a medical procedure, comprising selecting a medical procedure site on a patient, simultaneously applying a vibration to a first point and to a second point each adjacent to the medical procedure site, and performing the medical procedure at the medical procedure site.

Particular embodiments may comprise one or more of the following features. The method may further comprise maintaining the vibration adjacent to the medical procedure site for at least 15 seconds. The method may further comprise applying a force to the patient adjacent to the medical procedure site and applying the vibration in response to the force rising above a threshold value. The method may further comprise measuring a length of time that the force remains above the threshold value and only applying the vibration adjacent to the medical procedure site after the force remains above the threshold value for at least two seconds. The method may further comprise stopping the vibration when the force lowers past the threshold value. The threshold value is at least 3.5 Newtons. The method may further comprise applying the vibration at the first point in a first direction and applying the vibration at the second point in a second direction different from the first direction. The medical procedure site may be positioned between the first point and the second point. The vibration may have a frequency between 100 Hz and 200 Hz.

The foregoing and other aspects, features, and advantages will be apparent from the specification, drawings, and the claims.

### BRIEF DESCRIPTION OF THE DRAWINGS

Implementations will hereinafter be described in conjunction with the appended and/or included drawings, where like designations denote like elements, and:

FIG. 1 is a perspective view of a vibrating anesthesia device;

FIG. 2 is a perspective view of the first finger cuff and the second finger cuff of the vibrating anesthesia device shown in FIG. 1;

FIG. 3 is a perspective view of the first finger cuff and the second finger cuff shown in FIG. 1 with sections cut away to expose the interiors;

FIG. 4 is a perspective view of the first finger cuff shown in FIG. 1 with a section cut away to expose the interior;

FIG. 5 is a perspective view of the second finger cuff shown in FIG. 1 with a section cut away to expose the interior;

FIG. 6 is a top view of the first finger cuff shown in FIG. 1;

FIG. 7 is a cross section view of the first finger cuff taken from line 7-7 in FIG. 6;

FIG. 8 is a top view of the second finger cuff shown in FIG. 1;

FIG. 9 is a cross section view of the second finger cuff taken from line 9-9 in FIG. 8;

FIG. 10 is a perspective view of the control unit of the vibrating anesthesia device shown in FIG. 1;

FIG. 11 is a front exploded view of the control unit shown in FIG. 10;

FIG. 12 is a rear exploded view of the control unit shown in FIG. 10;

FIG. 13 is a process diagram illustrating a method of using the vibrating anesthesia device; and

FIG. 14 is a process diagram illustrating a method of reducing pain during a medical procedure.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The present disclosure is related to a vibrating anesthesia device 100. The vibrating anesthesia device 100 is configured to provide an anesthetic effect during a medical procedure such as an injection, an incision, a blood draw, placing or removing stitches, or some other medical procedure. The vibrating anesthesia device 100 increases the sensory input in a particular location on the patient, which helps to inhibit pain fiber activity and reduces the perception of pain. In particular, the vibrating anesthesia device 100 provides a physical stimulus of vibrations to provide this effect. Thus, the vibrating anesthesia device is configured to be worn by the user, who may a doctor, nurse, or other medical professional, and is configured to vibrate the patient at and/or around a medical procedure site to provide a local anesthetic effect.

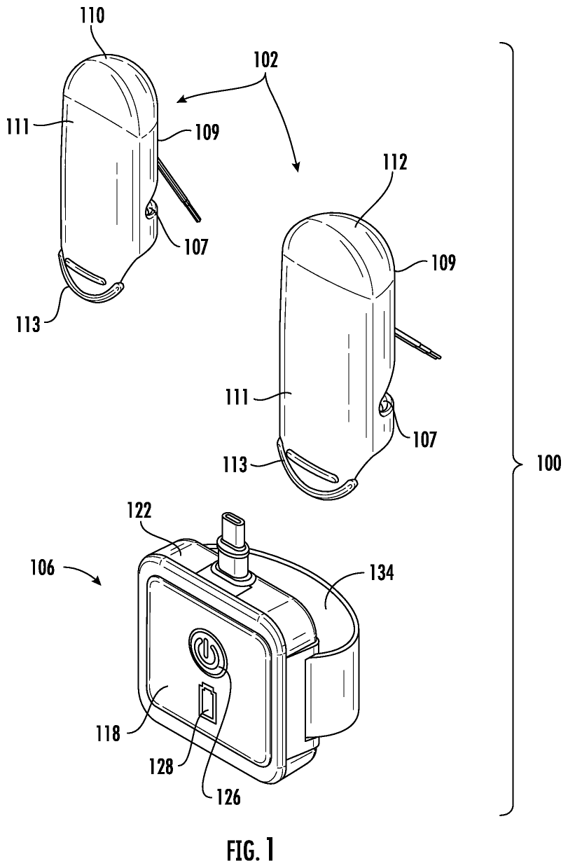

The vibrating anesthesia device 100 may comprise finger cuffs 102, a pressure switch 104, and a control unit 106, as shown in FIG. 1. In different embodiments, different numbers of finger cuffs 102 may be used. For example, the vibrating anesthesia device 100 may comprise one finger cuff 102, two finger cuffs 102, at least two finger cuffs 102, one or more finger cuffs 102, or any number of fingers cuffs 102.

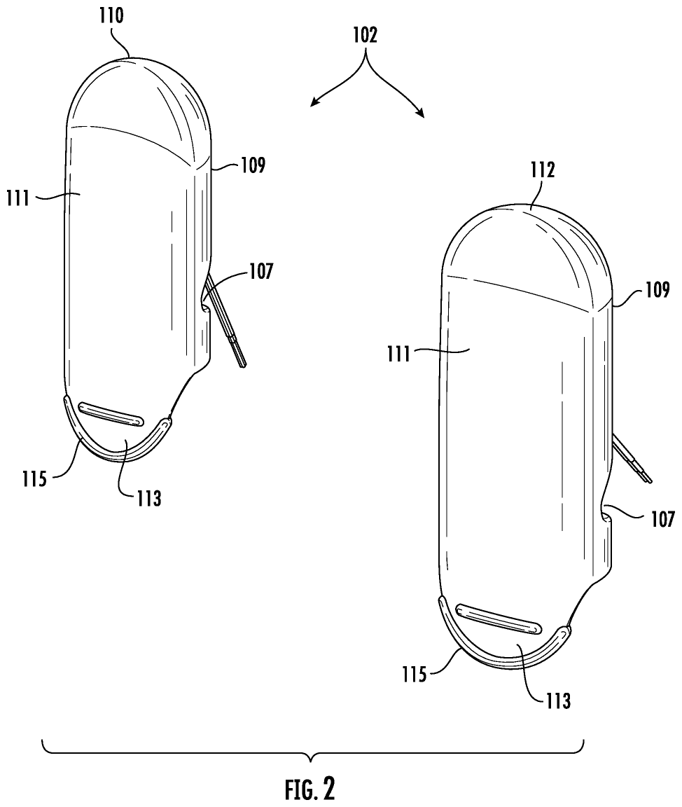

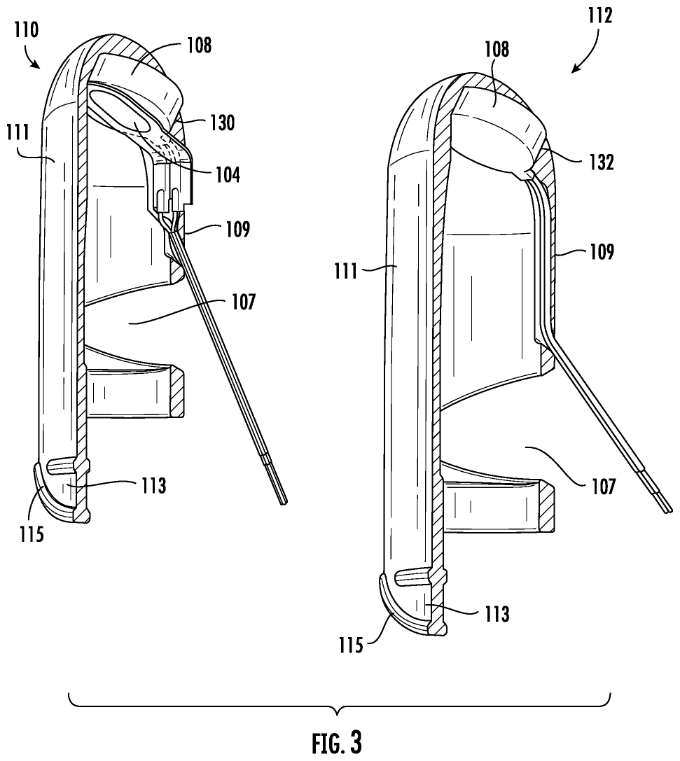

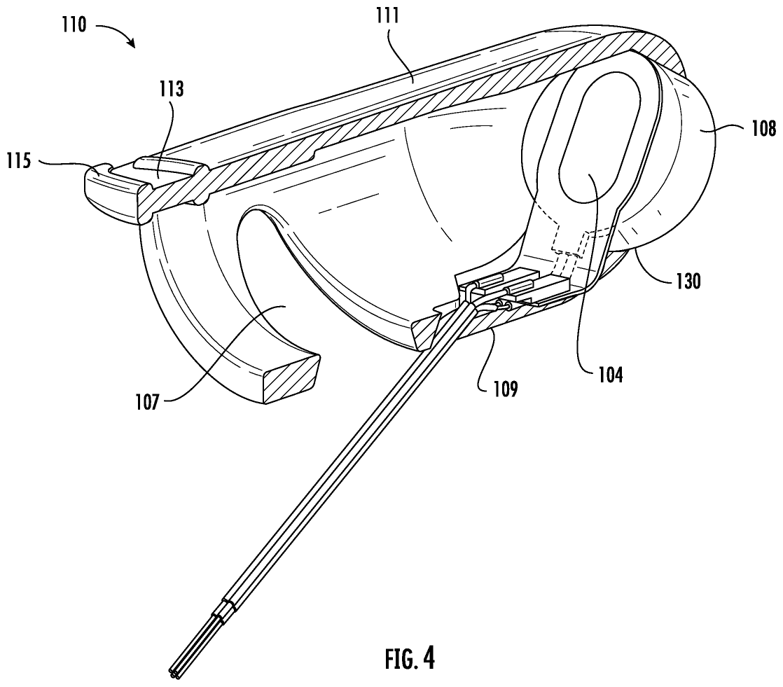

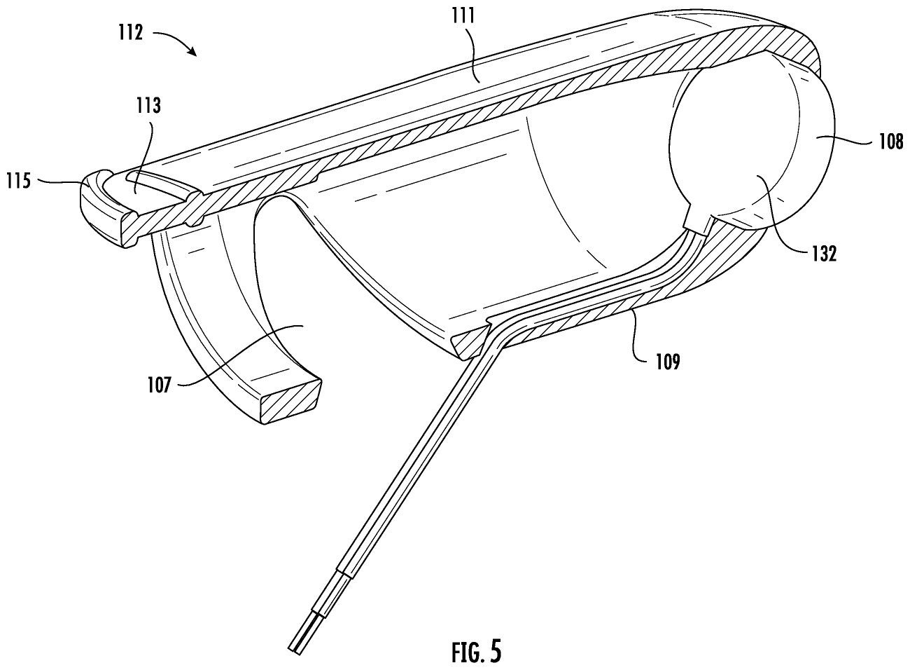

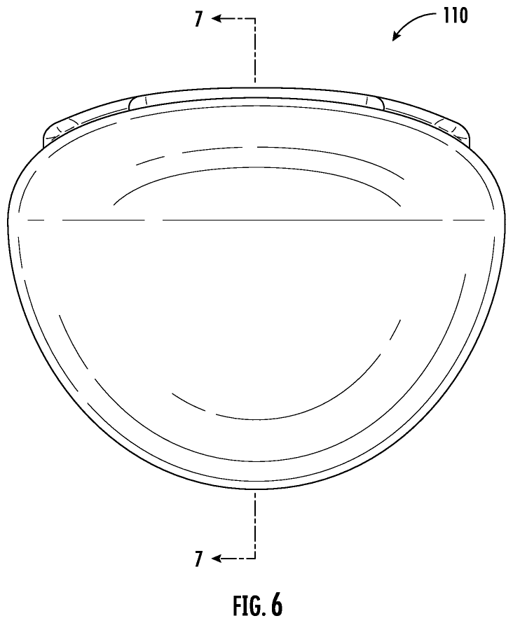

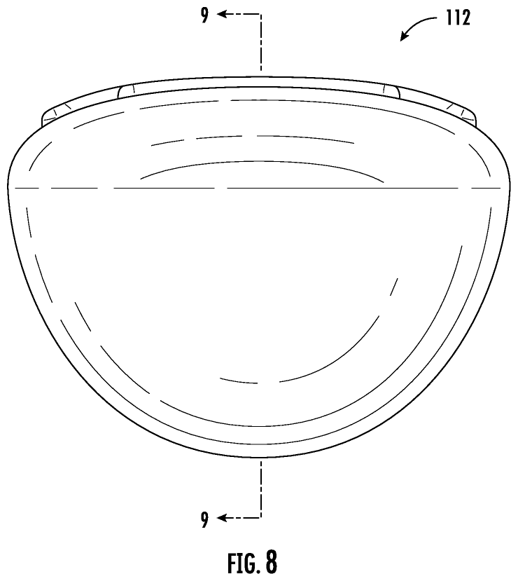

Turning to FIGS. 2-9, each finger cuff 102 is configured to fit onto a finger of a user. Each finger cuff 102 may have a vibrator motor 108. However, in some embodiments, there may be some finger cuffs 102 that do not include a vibrator motor 108. In particular embodiments, at least one of the one or more finger cuffs 102 has a vibrator motor 108. The vibrator motor 108 may be configured to be positioned adjacent to a fingertip of the user when the finger cuff 102 is positioned on the finger of the user. Thus, when in use, the user can position the vibrator motor 108 in a desired location, such as adjacent to a medical procedure site as discussed in more detail below, by positioning the user's fingertips in the desired location. The finger cuffs 102 may comprise a first finger cuff 110 and a second finger cuff 112. The first finger cuff 110 may be sized and shaped to fit on an index finger of the user and the second finger cuff 112 may be sized and shaped to fit on a thumb of the user.

Each finger cuff 102 may also have an open slit 107 that is configured to facilitate bending of the finger cuff 102. This allows the finger cuff 102 to be long enough to fit over a knuckle of the finger, which helps to keep the finger cuff 102 on the finger, without compromising the dexterity of the finger. The slit 107 may be positioned on a first side 109 of the finger cuff 102 opposite a second side 111, where the finger cuff 102 is configured to be worn with the first side 109 on the palmar surface of the user's hand and the second side 111 on the knuckle of the user. Each finger cuff 102 may also have a pull tab 113 that is configured to facilitate insertion of the finger into the finger cuff 102 by providing a tab for the user to pull on. The pull tab 113 may have at least one ridge 115 to improve the grip of the user on the pull tab 113.

In some embodiments, the finger cuffs 102 are permanently electrically coupled to the control unit 106. In other embodiments, the finger cuffs 102 are detachable from the control unit 106 to allow additional components to be plugged into the control unit 106. For example, a vibration probe may be implemented with a different form or function from the finger cuffs 102. Additionally, the finger cuffs 102 or other components may include additional functionality such as lighting, video, air pressure, etc. to enable the user to further improve the patient experience.

The pressure switch 104 is positioned within one of the finger cuffs 102. For example, the pressure switch 104 may be positioned within the first finger cuff 110 that is designed to be worn on the index finger of the user. The pressure switch 104 may be positioned such that, when the finger cuff 102 with the pressure switch 104 is worn, the pressure switch 104 can be activated by pressure from the tip of the finger or from the pad of the finger. The pressure switch 104 is configured to sense a force or pressure exerted on the pressure switch 104. The pressure switch 104 may be a force-sensitive resistor. This allows the pressure switch 104 to sense the force exerted on the pressure switch 104 indirectly by sensing the change of resistance of the pressure switch 104 as pressure or force is applied to the pressure switch 104. When at rest, the pressure switch 104 may have a high electrical resistance such that no current flows. When a force is applied, the resistance lowers such that some small current flows. In embodiments with a force-sensitive resistor, the pressure switch 104 may have a specification of <10 kΩ at 0.6 kg pressure. This means that when the resistance of the pressure switch 104 drops to a value of 10 kΩ, it is known that a “force” of 0.6 kg is being applied. In particular embodiments, the pressure switch 104 may have a specification of <10 kΩ at 0.3 kg pressure. Any other resistance and corresponding pressure may be selected. Applicant notes that sensor specifications frequently use mass and force interchangeably, with the understanding that on Earth's surface, one kilogram is equivalent to 9.8 Newtons of force. Similarly, force and pressure may also be used interchangeably, as discussed in more detail below. In particular, when the pressure switch 104 is a force-sensitive resistor, the area of the sensor is always known, and a conversion between pressure and force is a simple calculation.

Thus, while this disclosure may at times specifically refer to a force or to a pressure with respect to the pressure switch 104, it will be apparent to one of skill in the art that either value can be sensed and measured, and that one value can be calculated based on the other. Thus, for the purposes of this disclosure, force and pressure should be considered interchangeable, in particular because the force and/or pressure is only relevant in so far as to detect when the user desires to turn on the vibrating anesthesia device 100. In other words, the vibrating anesthesia device 100 is configured to be activated when the user wears the vibrating anesthesia device 100 and presses against the patient. This can be done both by sensing a force that rises above a threshold value or by sensing a pressure that rises above a threshold value, and these are considered to be equivalents. The advantage of such an activation method is that the device 100 can be turned on and off without requiring that a separate switch or button be pressed. The user can simply press the device 100 against the desired location and the device 100 automatically turns on, as described in more detail below.

The control unit 106 may be electrically and/or communicatively coupled to the pressure switch 104 and to each of the vibrator motors 108. The control unit 106 is configured to provide power to the pressure switch 104 so that the force exerted on the pressure switch 104 can be sensed. The control unit 106 is also configured to monitor the force exerted on the pressure switch 104. When the force exerted on the pressure switch 104 exceeds a threshold value, the control unit 106 is configured to turn on each of the vibrator motors 108. The threshold value may be at least 2 Newtons, at least 3 Newtons, at least 3.5 Newtons, at least 4 Newtons, at least 5 Newtons, or any other value. In some embodiments, the control unit 106 is configured to turn on each of the vibrator motors 108 only after the force exerted on the pressure switch 104 exceeds the threshold value for a predetermined period of time. The predetermined period of time may be at least two seconds in some embodiments. In other embodiments, the predetermined period of time may be 0.5 seconds, 1 second, 1.5 seconds, 2.5 seconds, 3 seconds, or any other period of time. This helps to avoid the vibrator motors 108 turning on unintentionally when the force very briefly exceeds the threshold value. Similarly, the control unit 106 may be configured to turn off each of the vibrator motors 108 only after the force exerted on the pressure switch 104 lowers past the threshold value for a predetermined period of time. This helps to avoid the vibrator motors 108 turning off unintentionally.

When turned on, the vibrator motors 108 are configured to vibrate. In some embodiments, the vibrator motors 108 are configured to vibrate at a frequency between 100 Hertz and 200 Hertz (Hz). In particular embodiments, the vibrator motors 108 are configured to vibrate at a frequency between 10 Hz and 200 Hz, or between 1 Hz and 1 kHz. Frequencies within these ranges can provide a local anesthetic effect when applied to a patient. The vibrator motors 108 may also be configured to have an adjustable motor impulse rate, and thus may be controlled by the control unit 106 to activate intermittently or at irregular intervals with pre-set or user adjustments. This, along with variable power that can be supplied to the vibrator motors 108, allows for a wider range of sensory vibrations due to adjustment of the frequency, impulse, and power.

When the finger cuffs 102 are worn on the fingers of the user and the user presses against a patient with the finger cuffs 102, the pressure switch 104 senses or detects a force exerted on the pressure switch 104. When this force exceeds the threshold value, the vibrator motors 108 turn on and begin vibrating. Because the finger cuffs 102 are pressed against the patient, this applies vibrations to the patient. In this way, the vibrator motors 108 are configured to provide a local anesthetic effect to the patient by vibrating when turned on. Having more than one vibrator motor 108 may be advantageous because the vibrator motors 108 can be placed at different locations around a medical procedure site. For example, when the finger cuffs 102 with vibrator motors 108 are on the user's index finger and thumb, these two fingers can be placed on either side of the medical procedure site, with the medical procedure site between the two fingers. The index finger and thumb can therefore be used to properly stretch or position the medical procedure site to for the medical procedure, such as to facilitate insertion of the needle during an injection, and also apply vibrations surrounding the medical procedure site so that the local anesthetic effect can be properly applied to the area surrounding the medical procedure site.

As part of the process for using the vibrating anesthesia device 100, the finger cuffs 102 may be moved and positioned in different configurations depending on the specific situation. For example, when inserting a needle into the abdomen area, a pinching motion may be used between two finger cuffs 102. In other locations that are flatter, the user may simply press down with the finger cuffs 102 instead of pinching. In other areas, it may be advantageous to press down in two completely different directions, such as when inserting a needle into the eyebrow, where the thumb is pressed underneath the orbital rim and the index finger is pressed above the eyebrow.

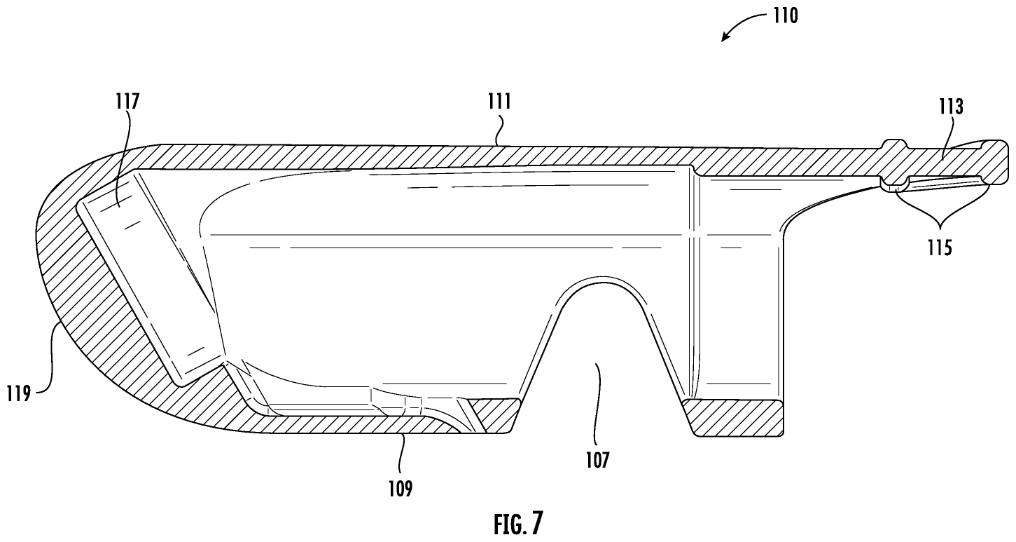

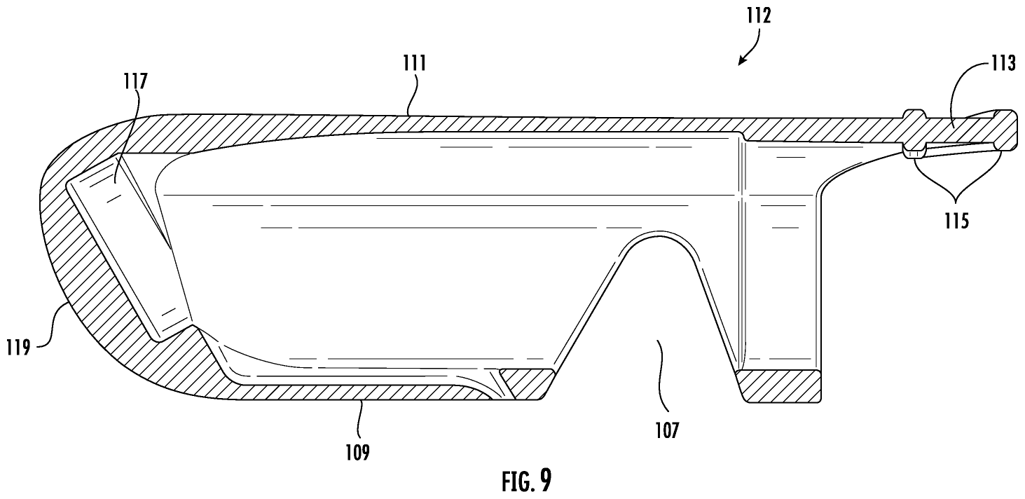

As noted above, the finger cuffs 102 may have the vibrator motors 108 positioned within the finger cuffs 102. In some embodiments, the finger cuffs 102 may include a cavity 115 inside of the finger cuff 102 near the tip 117 of the finger cuff 102 (see FIGS. 7 and 9). The cavity 115 is configured to provide a space for the vibrator motor 108 within the finger cuff 102 so that the finger cuff 102 still properly fits the finger of the user despite the presence of the vibrator 108 within the finger cuff 102.

To be clear regarding the present disclosure, while the figures show an embodiment where all of the finger cuffs 102 include a vibrator motor 108 and one of the finger cuffs 102 includes a pressure switch 104, alternative embodiments may have some finger cuffs 102 without a vibrator motor 108 and there may be more than one pressure switch 104. Thus, while in many embodiments, the pressure switch 104 is paired with a vibrator motor 108, in some embodiments, the pressure switch 104 may be alone, without a vibrator motor 108. Additionally, in embodiments with more than one pressure switch 104, the vibrator motors 108 may be independently operable such that some vibrator motors 108 can be turned on without turning on at least one of the other vibrator motors 108.

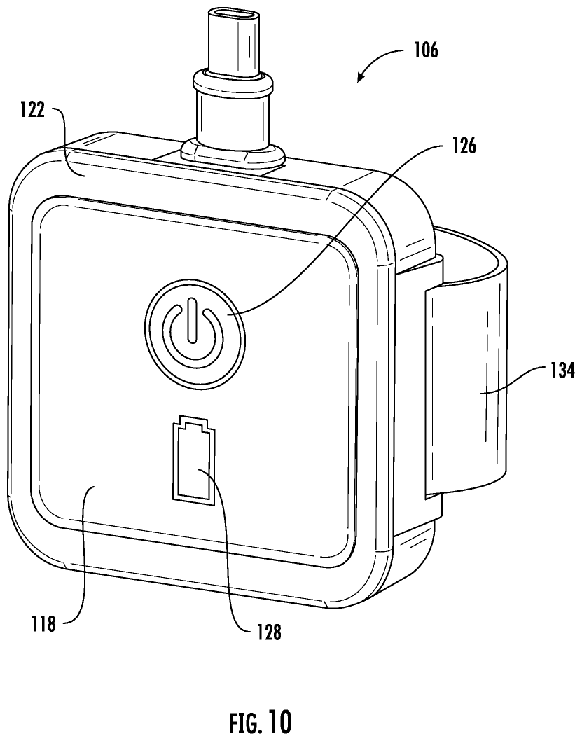

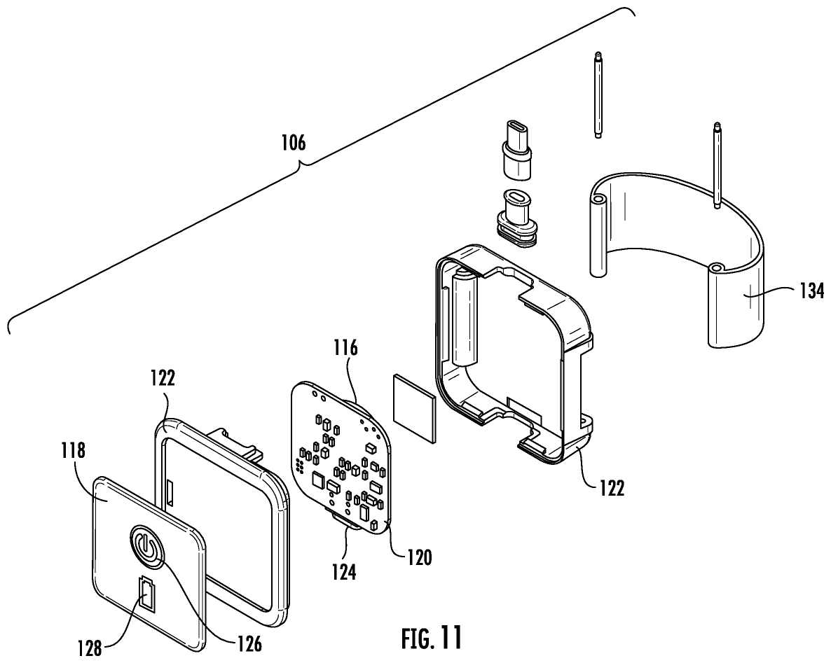

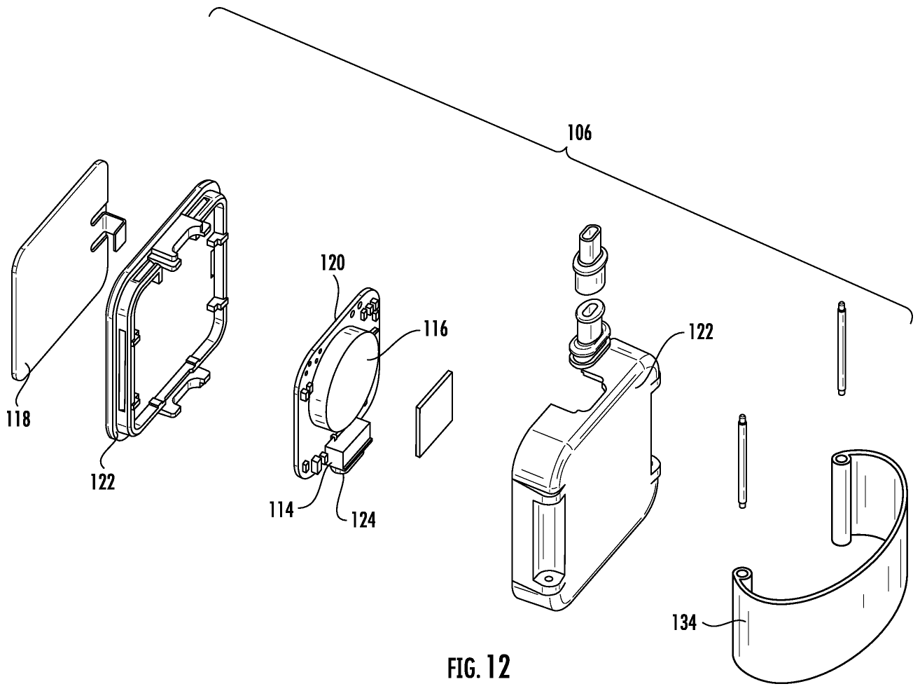

Turning to FIGS. 10-12, the control unit 106 may be configured to attach to the wrist of the user. For example, the control unit 106 may have a wrist band 134 configured to wrap around the wrist of the user. The wrist band 134 may be formed from elastic to facilitate attachment of the wrist band 134 to the user's body. Attaching the control unit 106 to the wrist or other body part of the user allows some of the components of the vibrating anesthesia device 100 to be positioned away from the fingers of the user and thus provides more space for such components without interfering with the use and dexterity of the user's fingers or with placement of gloves over the hands of the user. The control unit 106 could also be attached to other body parts of the user or could be positioned away from the body of the user. The control unit 106 may have a power source 114, a controller 116, and an interface 118. The power source 114 may be a battery. The power source 114 and the controller 116 may be integrated into a printed circuit board (PCB) 120. The power source 114 may be rechargeable. The control unit 106 may be housed in a housing 122, and the housing 122 may have a charging port 124 configured to receive a charging cord to recharge the power source 114. The charging port 124 may be a micro-USB charging port or any other charging port. The interface 118 is exposed through the housing 122 and is configured to allow the user to control the vibrating anesthesia device 100. For example, the interface 118 may provide a power button 126 to switch the vibrating anesthesia device 100 on and off. The interface 118 may also have a battery indicator 128 to show a level of charge remaining in the power source 114.

The vibrating anesthesia device 100 may also comprise a glove (not shown). The glove is configured to fit over a hand of the user and create a barrier between the vibrating anesthesia device 100 and the patient. This allows the vibrating anesthesia device 100 to be used for multiple patients without requiring that the vibrating anesthesia device 100 be sanitized between uses. Instead, only the glove can be replaced or sanitized. In embodiments where the vibrating anesthesia device 100 has the first finger cuff 110 worn on the index finger and the second finger cuff 112 worn on the thumb, the glove may be sized differently for these fingers to accommodate the vibrating anesthesia device 100. For example, an index finger of the glove may be sized to fit the first finger cuff 110 and the index finger of the user and a thumb of the glove may be sized to fit the second finger cuff 112 and the thumb of the user. Alternatively, a typical glove may be used to provide the barrier mentioned above. In such a scenario, the user may opt to wear a glove that is one size up from the user's typical size to allow room for the device within the glove.

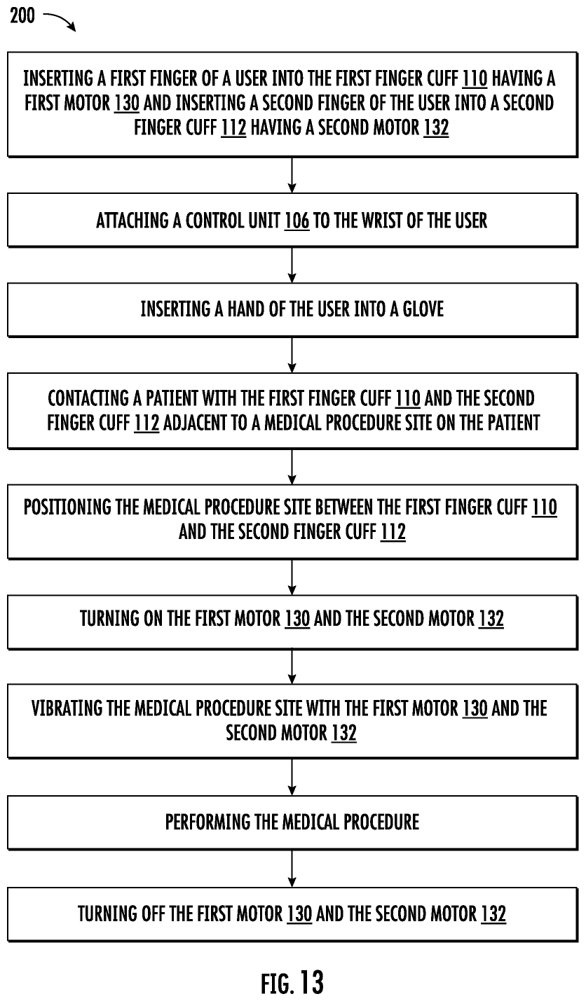

The present disclosure is also related to a method 200 of using the vibrating anesthesia device 100 disclosed above, illustrated in FIG. 13. The method 200 may comprise inserting a first finger of a user into the first finger cuff 110 having a first motor 130 and inserting a second finger of the user into a second finger cuff 112 having a second motor 132. The first motor 130 and the second motor 132 may both be vibrator motors 108 as described above. The method 200 may further comprise attaching a control unit 106 to the wrist of the user and/or inserting a hand of the user into a glove. The method 200 may also comprise contacting a patient with the first finger cuff 110 and the second finger cuff 112 adjacent to a medical procedure site on the patient. The medical procedure site is a location on the patient that has been designated for a medical procedure. For example, the medical procedure site may be an insertion site that has been designated for insertion of a needle, such as during an injection, a shot, or a blood draw, or may be an incision site or the site of an injury, such as a laceration. Contacting the patient adjacent to the medical procedure site may comprise positioning the medical procedure site between the first finger cuff 110 and the second finger cuff 112.

The method 200 may also comprise turning on the first motor 130 and the second motor 132, vibrating the medical procedure site with the first motor 130 and the second motor 132, performing the medical procedure at the medical procedure site, such as inserting a needle into the medical procedure site, and turning off the first motor 130 and the second motor 132. Vibrating the medical procedure site may comprise vibrating the medical procedure site for at least 15 seconds or at least 30 seconds and may comprise vibrating the medical procedure site at a frequency between 100 Hz and 200 Hz or between 1 Hz and 1 kHz. In the method 200, the first finger of the user may be an index finger and/or the second finger may be a thumb of the user. Turning on the first motor 130 and the second motor 132 may be in response to applying a force above a threshold value to the patient adjacent to the medical procedure site. The threshold value may be at least 0.5 Newtons, at least 1 Newton, at least 3.5 Newtons, or at least 5 Newtons.

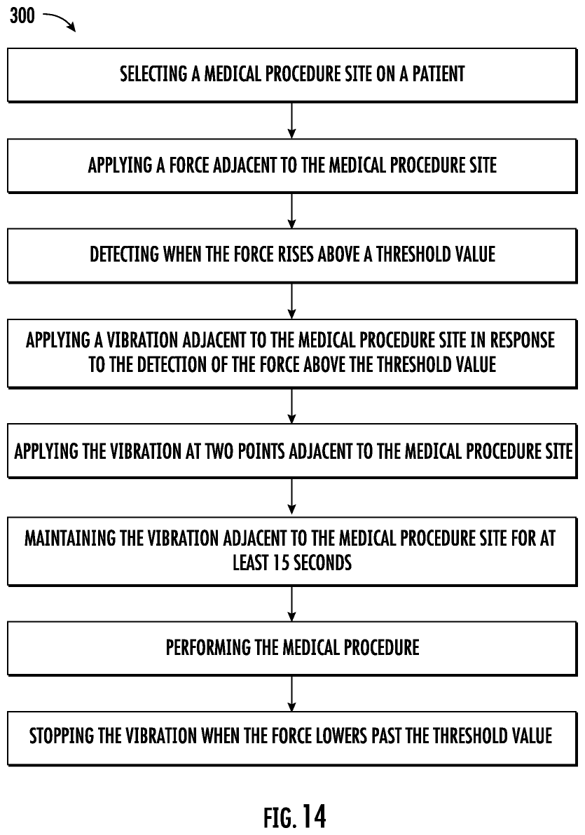

The present disclosure is also related to a method 300 of reducing pain during a medical procedure, illustrated in FIG. 14. The method 300 may comprise selecting a medical procedure site on a patient, applying a force to the patient adjacent to the medical procedure site, detecting when the force rises above a threshold value, applying a vibration adjacent to the medical procedure site in response to the force rising above the threshold value, and performing the medical procedure at the medical procedure site. The method 300 may also comprise maintaining the vibration adjacent to the medical procedure site for at least 15 seconds or at least 30 seconds. In method 300, a length of time that the force remains above the threshold value may be measured, and the vibration adjacent to the medical procedure site may be applied only after the force remains above the threshold value for at least half a second, at least 1 second, or at least two seconds. Applying the vibration adjacent to the medical procedure site may comprise applying the vibration at two points adjacent to the medical procedure site. The medical procedure site may be positioned between the two points and the vibration at the first point may be applied in a first direction while the vibration at the second point may be applied in a second direction different from the first direction. The method 300 may also comprise stopping the vibration when the force lowers past the threshold value. The vibration may have a frequency between 100 Hz and 200 Hz or between 1 Hz and 1 kHz, and the threshold value may be at least 0.5 Newtons, at least 1 Newton, at least 3.5 Newtons, or at least 5 Newtons.

The method 300 may also comprise selecting a medical procedure site on a patient, simultaneously applying a vibration to a first point and to a second point each adjacent to the medical procedure site, and performing the medical procedure at the medical procedure site. The method may also comprise maintaining the vibration adjacent to the medical procedure site for at least 15 seconds. The method may further comprise applying a force to the patient adjacent to the medical procedure site and applying the vibration in response to the force rising above a threshold value, measuring a length of time that the force remains above the threshold value and only applying the vibration adjacent to the medical procedure site after the force remains above the threshold value for at least two seconds, and/or stopping the vibration when the force lowers past the threshold value. As noted above, the threshold value may be at least 3.5 Newtons. Applying the vibration at the first point may be done in a first direction while applying the vibration at the second point may be done in a second direction different from the first direction. The medical procedure site may be positioned between the first point and the second point, and the vibration may have a frequency between 100 Hz and 200 Hz.

Many additional implementations are possible. Further implementations are within the CLAIMS.

It will be understood that implementations of the anesthesia device include but are not limited to the specific components disclosed herein, as virtually any components consistent with the intended operation of various anesthesia devices may be utilized. Accordingly, for example, it should be understood that, while the drawings and accompanying text show and describe particular anesthesia device implementations, any such implementation may comprise any shape, size, style, type, model, version, class, grade, measurement, concentration, material, weight, quantity, and/or the like consistent with the intended operation of anesthesia devices.

The concepts disclosed herein are not limited to the specific anesthesia devices shown herein. For example, it is specifically contemplated that the components included in particular anesthesia devices may be formed of any of many different types of materials or combinations that can readily be formed into shaped objects and that are consistent with the intended operation of the anesthesia device. For example, the components may be formed of: rubbers (synthetic and/or natural) and/or other like materials; glasses (such as fiberglass), carbon-fiber, aramid-fiber, any combination therefore, and/or other like materials; elastomers and/or other like materials; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; metals, such as zinc, magnesium, titanium, copper, iron, steel, carbon steel, alloy steel, tool steel, stainless steel, spring steel, aluminum, and/or other like materials; and/or any combination of the foregoing.

Furthermore, anesthesia devices may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular anesthesia device implementations, it should be readily apparent that a number of modifications may be made without departing from the spirit thereof and that these implementations may be applied to other implementations disclosed or undisclosed. The presently disclosed anesthesia devices are, therefore, to be considered in all respects as illustrative and not restrictive.

I/We claim:

1. A vibrating anesthesia device, comprising:
at least two flexible finger cuffs each configured to fit onto a finger of a user, each of the at least two finger cuffs having a vibrator motor configured to be positioned adjacent a fingertip of the user when the finger cuff is positioned on the finger of the user, wherein a first finger cuff of the at least two finger cuffs is sized and shaped to fit on an index finger of the user and a second finger cuff of the at least two finger cuffs is sized and shaped to fit on a thumb of the user;
a pressure switch positioned within the first finger cuff, wherein the pressure switch is configured to sense a force exerted on the pressure switch; and
a control unit configured to attach to a wrist of the user, the control unit having a power source, a controller, and an interface configured to allow the user to control the vibrating anesthesia device, wherein the control unit is electrically coupled to the pressure switch and to each of the vibrator motors and wherein the control unit is configured to provide power to the pressure switch, monitor the force exerted on the pressure switch, and turn on each of the vibrator motors of the at least two finger cuffs when the force exerted on the pressure switch exceeds a threshold value;
wherein, when turned on, the vibrator motors are configured to vibrate and provide a local anesthetic effect to a patient.

2. The vibrating anesthesia device of claim 1, further comprising a glove configured to fit over a hand of the user and create a barrier between the vibrating anesthesia device and the patient, wherein an index finger of the glove is sized to fit the first finger cuff and the index finger of the user, and a thumb of the glove is sized to fit the second finger cuff and the thumb of the user.

3. The vibrating anesthesia device of claim 1, wherein the control unit is configured to turn on each of the vibrator motors only after the force exerted on the pressure switch exceeds the threshold value for at least two seconds.

4. The vibrating anesthesia device of claim 1, wherein the vibrator motors are configured to vibrate at a frequency between 100 Hz and 200 Hz.

5. The vibrating anesthesia device of claim 1, wherein the threshold value is at least 3.5 Newtons.

6. A vibrating anesthesia device, comprising:
a first finger cuff configured to fit onto a finger of a user, the first finger cuff having a vibrator motor configured to be positioned adjacent a fingertip of the user when the finger cuff is positioned on the finger of the user;
a pressure switch positioned within the first finger cuff, wherein the pressure switch is configured to sense a force exerted on the pressure switch; and
a control unit configured to attach to a wrist of the user, wherein the control unit is electrically coupled to the pressure switch and to the vibrator motor and wherein the control unit is configured to provide power to the pressure switch, monitor the force exerted on the pressure switch, and turn on the vibrator motor when the force exerted on the pressure switch exceeds a threshold value;
wherein, when turned on, the vibrator motor is configured to vibrate and provide a local anesthetic effect to a patient.

7. The vibrating anesthesia device of claim 6, further comprising a second finger cuff configured to fit onto a second finger of the user, the second finger cuff having a vibrator motor, wherein the control unit is configured to turn on the vibrator motor of the second finger cuff with the vibrator motor of the first finger cuff.

8. The vibrating anesthesia device of claim 7, wherein the first finger cuff is sized and shaped to fit on an index finger of the user and the second finger cuff is sized and shaped to fit on a thumb of the user.

9. The vibrating anesthesia device of claim 7, further comprising a glove configured to fit over a hand of the user and create a barrier between the vibrating anesthesia device and the patient, wherein an index finger of the glove is sized to fit the first finger cuff and the index finger of the user, and a thumb of the glove is sized to fit the second finger cuff and the thumb of the user.

10. The vibrating anesthesia device of claim 6, wherein the control unit is configured to turn on the vibrator motor only after the force exerted on the pressure switch exceeds the threshold value for at least two seconds.

11. The vibrating anesthesia device of claim 6, wherein the vibrator motor is configured to vibrate at a frequency between 100 Hz and 200 Hz.

12. The vibrating anesthesia device of claim 6, wherein the threshold value is at least 3.5 Newtons.

13. A method of reducing pain during a medical procedure, comprising:
selecting a medical procedure site on a patient;
applying a force to the patient adjacent to the medical procedure site;
applying a vibration adjacent to the medical procedure site in response to the force rising above a threshold value; and
performing the medical procedure at the medical procedure site.

14. The method of claim 13, further comprising maintaining the vibration adjacent to the medical procedure site for at least 15 seconds.

15. The method of claim 13, further comprising measuring a length of time that the force remains above the threshold value and only applying the vibration adjacent to the medical procedure site after the force remains above the threshold value for at least two seconds.

16. The method of claim 13, wherein applying the vibration adjacent to the medical procedure site comprises applying the vibration at two points adjacent to the medical procedure site.

17. The method of claim 16, further comprising applying the vibration at a first point of the two points in a first direction and applying the vibration at a second point of the two points in a second direction different from the first direction.

18. The method of claim 16, wherein the medical procedure site is positioned between the two points.

19. The method of claim 13, further comprising stopping the vibration when the force lowers past the threshold value.

20. The method of claim 13, wherein the vibration has a frequency between 100 Hz and 200 Hz.
